(19) United States
(12) Patent Application Publication
(10) Pub. No.: US 2025/0302849 A1
(43) Pub. Date: Oct. 2, 2025

(54) ORAL PAIN RELIEVER COMPOSITION, PHARMACEUTICAL FORMULATION, AND PROCESS FOR MAKING COMPOSITION AND FORMULATION INCLUDING CANNABIDIOL ISOLATE WITH SELECT ACTIVE INGREDIENTS

(71) Applicants: Michael Cavender, Morgantown, WV (US); Christopher Grose, Glenwood, WV (US)

(72) Inventors: Michael Cavender, Morgantown, WV (US); Christopher Grose, Glenwood, WV (US)

(21) Appl. No.: 19/095,315

(22) Filed: Mar. 31, 2025

Related U.S. Application Data
(60) Provisional application No. 63/572,310, filed on Mar. 31, 2024.

(51) Int. Cl. A61K 31/00; A61K 31/192; A61K 31/196; A61K 31/405; A61K 31/415; A61K 31/444; A61K 31/616

(52) U.S. Cl. CPC A61K 31/658; A61K 31/192; A61K 31/196; A61K 31/405; A61K 31/415; A61K 31/444; A61K 31/616

(57) ABSTRACT

A chemical composition for an oral pharmaceutical product is described having a therapeutically effective amount of one or more active pharmaceutical ingredients (API) selected from anti-inflammatory and/or analgesic and/or non-steroidal anti-inflammatory (NSAIDS) and/or antipyretic group(s) of ingredients utilized in combination with a cannabidiol (CBD) isolate.

### I. RELATED APPLICATIONS

This application claims the benefit of U.S. Provisional Application No. 63/572,310, filed on Mar. 31, 2024.

### II. FIELD OF THE INVENTION

The present application discloses and describes a pain reliever, and in particular, a composition and process of making the composition for an oral pharmaceutical application as a pain reliever.

### III. BACKGROUND OF THE INVENTION

Numerous attempts have been made to improve the management and administration of pain relief to individuals. Presently, considerable effort and attention is devoted toward pain management that effectively provides pain relief without contributing to and/or increasing the likelihood of long-term dependency and/or addiction.

Pain management is a critical aspect of healthcare, with oral pain relievers playing a significant role in alleviating discomfort associated with various conditions, including dental procedures, migraines, musculoskeletal injuries, and chronic pain disorders. Traditional oral analgesics, including nonsteroidal anti-inflammatory drugs (NSAIDs), acetaminophen, ibuprofen, and/or opioid-based medications, have been widely used for decades. However, these existing formulations present several challenges, including delayed onset of action, gastrointestinal side effects, potential for liver or kidney toxicity, and risks of dependency or abuse in the case of opioids.

Efforts to improve oral pain relief have focused on enhancing drug bioavailability, increasing the speed of pain relief, and minimizing adverse effects. Various strategies, such as modified-release formulations, nanoparticle delivery systems, and alternative active ingredients, have been explored. Despite these advancements, there remains an unmet need for an oral pain relief formulation that provides rapid and sustained analgesic effects while reducing the risk of side effects and misuse.

Cannabinoids are a diverse group of chemical compounds found in the cannabis plant, and they interact with the endocannabinoid system (ECS) in the human body. The ECS is responsible for regulating various physiological processes, including pain, mood, appetite, immune function, and memory. Cannabinoids can either mimic or influence the body's natural endocannabinoids, leading to a range of effects.

There are over 100 identified cannabinoids in cannabis. In particular, phytocannabinoids and synthetic cannabinoids are investigated for providing pain relief and anti-imflammatory effects, although the unpredictability raises concerns. The effectiveness of such methods are variable because of bioavailability, biodiversity of individuals, and various medical and non-medical interactions. In addition, short-term and long-term side effects raise additional concerns.

The present invention addresses these challenges by introducing an innovative oral pain relief composition that optimizes drug delivery and efficacy. Through novel formulation techniques and improved pharmacokinetic properties, this invention seeks to offer a safer, faster-acting, and more effective alternative to existing oral pain relievers. Accordingly, a need exists for improved compositions, delivery mechanisms, administration routes, and therapies for pain relief management.

### IV. SUMMARY OF THE INVENTION

In one embodiment, a process for forming a pharmaceutical tablet comprising the step of selecting one or more active ingredients from the group comprising an anti-inflammatory active ingredient, an analgesic active ingredient, an NSAID active ingredient, and an antipyretic active ingredient; combining the one or more active ingredients with cannabidiol isolate active ingredient, the process further comprising the steps of blending, granulating, drying, compressing, coating, and polishing the tablet.

In another embodiment, a pharmaceutical composition comprises cannabidiol isolate, present in an amount ranging from about 2% to about 35% by dry weight of the composition, one or more active ingredients selected from the group comprising naproxen, aspirin, diclofenac, celecoxib, etoricelecoxib, mefenamic acid, and indomethacin present in an amount ranging from about 65% to about 98% by dry weight of the composition, and one or more pharmaceutically acceptable excipients, stabilizers, or carriers, selected from the group consisting of starch, sucrose, gelatin, sugar alcohols, isomalt, cellulose derivatives, and polyvinylpyrrolidone, wherein the composition is formulated for oral administration.

In the pharmaceutical composition, in one alternative, the dosage form is by tablet form.

In the pharmaceutical composition, and in another alternative, the dosage form is by capsule form.

In another embodiment, a method of treating pain in a patient in need thereof, the method comprising the step of administering to the patient an effective amount of cannabidiol isolate and one or more of one or more active ingredients selected from the group comprising naproxen, aspirin, diclofenac, celecoxib, etoricelecoxib, mefenamic acid, and indomethacin, wherein cannabidiol isolate comprises 2% to 35% of the total weight of the active ingredient combination and the one or more of one or more active ingredients selected from the group comprising naproxen, aspirin, diclofenac, celecoxib, etoricelecoxib, mefenamic acid, and indomethacin comprises 65% to 98% of the total weight of the active ingredient combination, via oral administration.

### VI. DETAILED DESCRIPTION OF THE EMBODIMENT(S)

It will be readily understood that the components of the present invention, as generally described and illustrated in the figures herein, may be arranged and designed in a wide variety of different configurations. Thus, the following detailed description of the embodiments, as represented in the attached figures, is not intended to limit the scope of the invention as claimed but is merely representative of selected embodiments of the invention.

The features, structures, or characteristics of the invention described throughout this specification may be combined in any suitable manner in one or more embodiments. For example, the usage of the phrases “example embodiments”, “some embodiments”, or other similar language, throughout this specification refers to the fact that a particular feature, structure, or characteristic described in connection with the embodiment may be included in at least one embodiment of the present invention. Thus, appearances of the phrases “example embodiments”, “in some embodiments”, “in other embodiments”, or other similar language, throughout this specification do not necessarily all refer to the same group of embodiments, and the described features, structures, or characteristics may be combined in any suitable manner in one or more embodiments.

According to at least one embodiment, and consistent with the description herein, a chemical composition for an oral pharmaceutical product is envisioned. The pharmaceutical comprises a therapeutically effective amount of one or more active pharmaceutical ingredients (API). The API(s) is/are selected from anti-inflammatory and/or analgesic and/or non-steroidal anti-inflammatory (NSAIDS) and/or antipyretic (fever reducing) group(s) of active ingredients utilized in combination with a cannabidiol (CBD) isolate.

It is envisioned that the chemical composition for the oral pharmaceutical product may comprise a variety of forms and from a variety of manufacturing processes, mainly targeting digestive tract as a route of administration, and including both solid and liquid forms. For example, one preferred embodiment comprises powder and/or similarly solid forms of the API(s) utilized. The substantially-powder and/or solid API forms may be formed into tablets via a compression process. Moreover, the substantially-powder and/or solid API forms may be formed into the ingredients disposed in a capsule. In such forms, it is envisioned that the API(s) utilized may include a binder excipient, and with the tablet form comprising API(s) and binder excipient inside a digestible shell, and with the capsule form comprising API(s) and binder excipient encapsulated inside a digestible hard-shell capsule. In a softgel form, the API(s) are use in combination with liquid carrier(s) and encapsulated inside a digestible softgel capsule. It is also envisioned that other forms may be utilized, including but not limited to various liquid suspensions, solutions, syrups, emulsions, and/or elixirs. It is also envisioned that the buccal and/or sublingual routes of administration may also be used, including such forms as medicated lozenges (also known as troche or pastille), orally disintegrating tablets, films, lollipops, gums, and/or tablets, as well as liquids including sprays and/or ointments.

Generally, the formulations described herein are chemical compositions defining a pharmaceutical having therapeutically effective amounts of one or more active pharmaceutical ingredients that promote pain relief in vivo. One of the APIs is CBD isolate, an odorless and tasteless crystalline solid or powder extracted from a cannabis plant. CBD isolate can boost the immune system and includes anti-inflammatory and/or analgesic properties for fighting inflammation and relieving pain. CBD isolate addresses the cannabinoid receptor pathway and the various cannabinoid receptors in the pathway. There are some indications that CBD isolate has influence over non-cannabinoid receptors for immune and/or analgesic response(s).

Such a composition comprises a binder excipient compound necessary to chemically and physically bind the API(s) and provide the necessary minimum mechanical strength to the composition for forming a tablet. The binder compound is easily absorbed by the body without appreciable in vivo effects.

A binder excipient may include a variety of compounds and/or polymers, such as starch, sucrose, gelatin, sugar alcohols, isomalt, cellulose derivatives, and polyvinylpyrrolidone. A binder excipient can affect the various properties of the combination, including solubility, release of active ingredient(s), compaction, and strength, either separately or in combination(s). A binder excipient improves disintegration, volume, dissolution, and bioavailability of the pharmaceutical.

Generally, the manufacturing process for forming a pharmaceutical tablet includes blending, granulation, drying, compression, coating, polishing, branding, quality assurance/quality control, and packaging. The overall process may include the consolidation of certain steps or the disaggregation of certain steps as appropriate for the materials, quantity (ies) of active ingredient(s) utilized, and other related concerns. In particular, each portion of the process will be briefly discussed.

The blending stage includes mixing the API(s) and excipient(s) into an industrial blender. The API(s) and excipient(s) are each powder, granules, and/or dry solid forms, or combinations thereof. The blending stage is necessary to produce a homogenous mixture of these dry powder/granule ingredients, and thereby distribute the API(s) as evenly across the blend as possible.

In the granulation stage, any powder ingredient(s) is/are converted into granules. The binder excipient(s) is/are added at the granulation stage. Because release of the API(s) is dependent upon granulation, this stage is adjusted to generate the appropriate size and shape of the granules.

Thereafter, the granules are subjected to a drying stage to remove excess solvents extant and remove excess moisture from the emerging composition. A plurality of dryer forms and/or devices may be utilized, including tray and/or bed dryer units. Moisture content is observed and threshold moisture content limits assist in reaching the appropriate moisture content level.

After drying, the granules are compressed into a tablet form. This may comprise one or more combinations of operations, including milling and/or mixing of the granules. Punches and dies are used to provide the exterior boundary of the tablet, wherein the dried granules representing the blend of API(s) and binder excipient(s) are injected or otherwise introduced into the chamber(s) formed by the punch(es) and die(s). A tablet press machine applies significant high pressure to the API(s) and binder excipient(s) material injected between the punch(es) and die(s). As noted above, this process physically forms the selected tablet shape while adding mechanical strength and influencing such properties as dissolution, disintegration, and bioavailability of the API(s).

After a quantity of tablets are formed, spraying and/or dipping method(s) may be used separately or in combination to apply a smooth, outer layer to the compressed solid material(s). The coating stage prepares the emerging tablet for formation of an outer coating to be applied. The coating provides additional mechanical strength and a protective exterior layer enveloping the API(s). Moreover, the coating allows for the organization's unique brand(s) and/or trademark(s) to be applied for convenient commercial identification. It is envisioned that the composition is designed for a variety of forms of delivery, including immediate release, controlled release, sustained release, and/or targeted delivery.

Subsequent to the coating stage, the tablets are polished to remove any surface imperfections and to provide as smooth a surface as practicable. Edible food grade inks are utilized to then impart the brand(s) and/or trademark(s) thereon. This stage allows the organization to visually distinguish itself. Moreover, the smooth, edible exterior coating removes and/or reduces the difficulty that many users may experience trying to swallow and otherwise ingest the tablet(s).

Once completed, the tablets are evaluated during the quality assurance/quality control stage. The tablets must meet specific production specifications, including the randomized testing of a multiplicity of characteristics and parameters. These tests include both the physical composition and the mechanical endurance of the tablets, as well as evaluating for homogeneity or uniformity. Once quality assurance/quality control is completed and the appropriate approval thresholds are met, the tablets are prepared for packaging and distribution.

It is envisioned that multiple formulations of anti-inflammatory, analgesic, and/or pain-relieving pharmaceutical medications may be produced from the general methodology described above. In particular, at least nine formulations are envisioned, covering combinations of anti-inflammatory compositions in combination with CBD isolate, combinations of analgesic compositions in combination with CBD isolate, combinations of non-steroidal anti-inflammatory compositions (NSAIDS) with CBD isolate, and/or combinations of and/or antipyretic (fever reducing) compositions with CBD isolate. It is also envisioned that additional subcombinations of compositions may be formed using anti-inflammatory, analgesic, NSAID compositions, and/or antipyretic with the CBD isolate. Each of the formulations envisioned are described in greater detail below.

In one embodiment, a composition comprising therapeutically effective amounts of naproxen and cannabidiol (CBD) isolate, the active pharmaceutical ingredients (API) for generating pain relieving effects in the human body when taken orally by an individual. As noted above, one or more binder excipient compounds is/are necessary to chemically and physically bind the naproxen and the CBD isolate active ingredients and provide necessary minimum mechanical strength to the composition for then forming as a tablet.

The combination for the aforementioned formulation may be represented by:

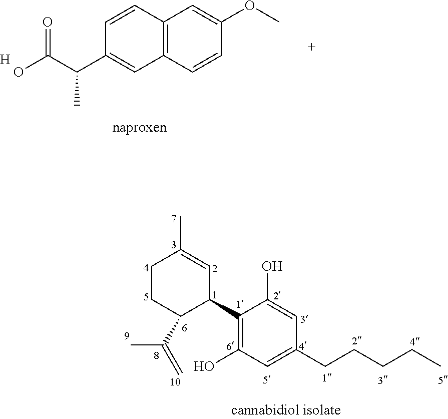

It is envisioned that the total dry volume of the combined formulations for a single dosage tablet or capsule comprises 220 mg of naproxen and 25 mg of CBD isolate. It is envisioned that a single dosage tablet or capsule may comprise lower or higher dry volume quantities of naproxen. However, a single dosage tablet or capsule has a maximum dry volume of 750 mg of naproxen. It is envisioned that adjustments from the 220 mg (naproxen) baseline includes an upward or downward proportional adjustment of the dry volume of CBD isolate, including for dry volume of 250 mg, 375 mg, 500 mg, and 550 mg as intermediate ranges between the 220 mg and 750 mg maximum for naproxen.

In another embodiment, a composition comprising therapeutically effective amounts of aspirin and cannabidiol (CBD) isolate, the active pharmaceutical ingredients (API) for generating pain relieving effects in the human body when taken orally by an individual. As noted above, one or more binder excipient compounds is/are necessary to chemically and physically bind the aspirin and the CBD isolate active ingredients and provide necessary minimum mechanical strength to the composition for then forming as a tablet.

The combination for the aforementioned formulation may be represented by:

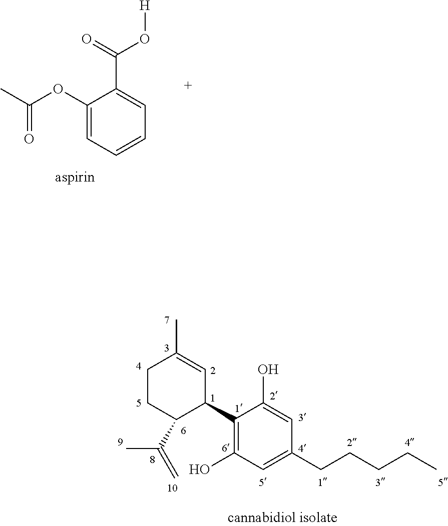 [SMILES: CC(=O)Oc1ccccc1C(=O)O ; C=C(C)[C@@H]1CCC(C)=C[C@H]1c1c(O)cc(CCCCC)cc1O]

It is envisioned that the total dry volume of the combined formulations for a single dosage tablet or capsule comprises 325 mg of aspirin and 25 mg of CBD isolate. It is envisioned that a single dosage tablet or capsule may comprise lower or higher dry volume quantities of aspirin. However, a single dosage tablet or capsule has a maximum dry volume of 650 mg of aspirin. It is envisioned that adjustments from the 325 mg (aspirin) baseline includes an upward or downward proportional adjustment of the dry volume of CBD isolate, including for dry volume in intermediate ranges between the 325 mg and 650 mg maximum for aspirin.

In another embodiment, a composition comprising therapeutically effective amounts of diclofenac and cannabidiol (CBD) isolate, the active pharmaceutical ingredients (API) for generating pain relieving effects in the human body when taken orally by an individual. As noted above, one or more binder excipient compounds is/are necessary to chemically and physically bind the diclofenac and the CBD isolate active ingredients and provide necessary minimum mechanical strength to the composition for then forming as a tablet.

The combination for the aforementioned formulation may be represented by:

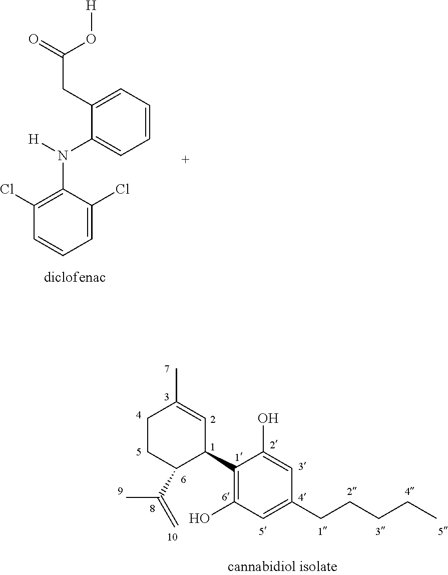 [SMILES: O=C(O)Cc1ccccc1Nc1c(Cl)cccc1Cl ; C=C(C)[C@@H]1CCC(C)=C[C@H]1c1c(O)cc(CCCCC)cc1O]

It is envisioned that the total dry volume of the combined formulations for a single dosage tablet or capsule comprises 50 mg of diclofenac and 25 mg of CBD isolate. It is envisioned that a single dosage tablet or capsule may comprise lower or higher dry volume quantities of diclofenac. However, a single dosage tablet or capsule has a maximum dry volume of 100 mg of diclofenac. It is envisioned that adjustments from the 50 mg (diclofenac) baseline includes an upward or downward proportional adjustment of the dry volume of CBD isolate, including for dry volume in intermediate ranges between the 50 mg and 100 mg maximum for diclofenac.

In another embodiment, a composition comprising therapeutically effective amounts of celecoxib and cannabidiol (CBD) isolate, the active pharmaceutical ingredients (API) for generating pain relieving effects in the human body when taken orally by an individual. As noted above, one or more binder excipient compounds is/are necessary to chemically and physically bind the celecoxib and the CBD isolate active ingredients and provide necessary minimum mechanical strength to the composition for then forming as a tablet.

The combination for the aforementioned formula may be represented by:

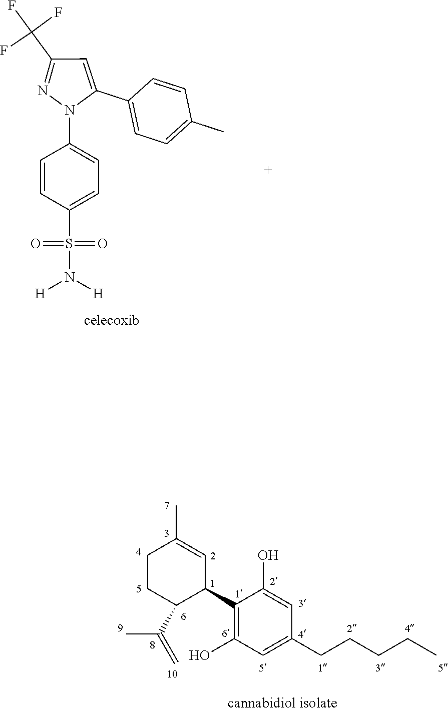 [SMILES: Cc1ccc(-c2cc(C(F)(F)F)nn2-c2ccc(S(N)(=O)=O)cc2)cc1 ; C=C(C)[C@@H]1CCC(C)=C[C@H]1c1c(O)cc(CCCCC)cc1O]

It is envisioned that the total dry volume of the combined formulations for a single dosage tablet or capsule comprises 100 mg of celecoxib and 25 mg of CBD isolate. It is envisioned that a single dosage tablet or capsule may comprise lower or higher dry volume quantities of celecoxib. However, a single dosage tablet or capsule has a maximum dry volume of 200 mg of celecoxib. It is envisioned that adjustments from the 100 mg (celecoxib) baseline includes an upward or downward proportional adjustment of the dry volume of CBD isolate, including for dry volume in intermediate ranges between the 100 mg and 200 mg maximum for celecoxib.

In another embodiment, a composition comprising therapeutically effective amounts of etoricoxib and cannabidiol (CBD) isolate, the active pharmaceutical ingredients (API) for generating pain relieving effects in the human body when taken orally by an individual. As noted above, one or more binder excipient compounds is/are necessary to chemically and physically bind the etoricoxib and the CBD isolate active ingredients and provide necessary minimum mechanical strength to the composition for then forming as a tablet.

The combination for the aforementioned formulation may be represented by:

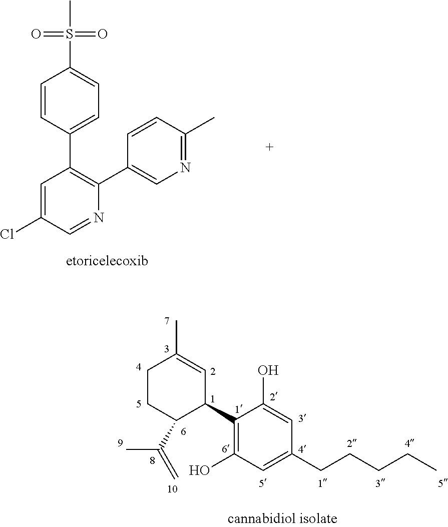 [SMILES: Cc1ccc(-c2ncc(Cl)cc2-c2ccc(S(C)(=O)=O)cc2)cn1 ; C=C(C)[C@@H]1CCC(C)=C[C@H]1c1c(O)cc(CCCCC)cc1O]

It is envisioned that the total dry volume of the combined formulations for a single dosage tablet or capsule comprises 90 mg of etoricelecoxib and 25 mg of CBD isolate. It is envisioned that a single dosage tablet or capsule may comprise lower or higher dry volume quantities of etoricelecoxib. However, a single dosage tablet or capsule has a maximum dry volume of 120 mg of etoricelecoxib. It is envisioned that adjustments from the 90 mg (etoricelecoxib) baseline includes an upward or downward proportional adjustment of the dry volume of CBD isolate, including for dry volume in intermediate ranges between the 60 mg and 120 mg maximum for etoricelecoxib.

In another embodiment, a composition comprising therapeutically effective amounts of mefenamic acid and cannabidiol (CBD) isolate, the active pharmaceutical ingredients (API) for generating pain relieving effects in the human body when taken orally by an individual. As noted above, one or more binder excipient compounds is/are necessary to chemically and physically bind the mefenamic acid and the CBD isolate active ingredients and provide necessary minimum mechanical strength to the composition for then forming as a tablet.

The combination for the aforementioned formulation may be represented by:

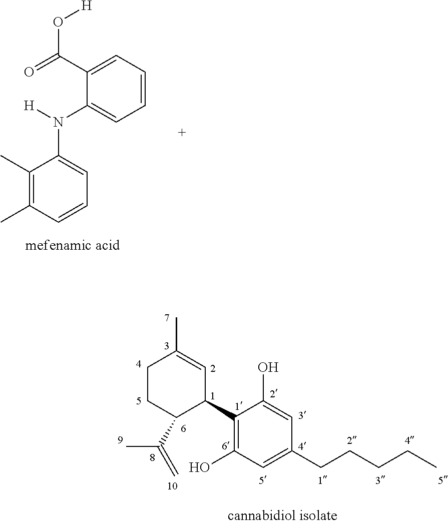 [SMILES: Cc1cccc(Nc2ccccc2C(=O)O)c1C ; C=C(C)[C@@H]1CCC(C)=C[C@H]1c1c(O)cc(CCCCC)cc1O]

It is envisioned that the total dry volume of the combined formulations for a single dosage tablet or capsule comprises 250 mg of mefenamic acid and 25 mg of CBD isolate. It is envisioned that a single dosage tablet or capsule may comprise lower or higher dry volume quantities of mefenamic acid. However, a single dosage tablet or capsule has a maximum dry volume of 200 mg of mefenamic acid. It is envisioned that adjustments from the 250 mg (mefenamic acid) baseline includes an upward or downward proportional adjustment of the dry volume of CBD isolate, including for dry volume in intermediate ranges.

In another embodiment, a composition comprising therapeutically effective amounts of indomethacin and cannabidiol (CBD) isolate, the active pharmaceutical ingredients (API) for generating pain relieving effects in the human body when taken orally by an individual. As noted above, one or more binder excipient compounds is/are necessary to chemically and physically bind the indomethacin and the CBD isolate active ingredients and provide necessary minimum mechanical strength to the composition for then forming as a tablet.

The combination for the aforementioned formulation may be represented by:

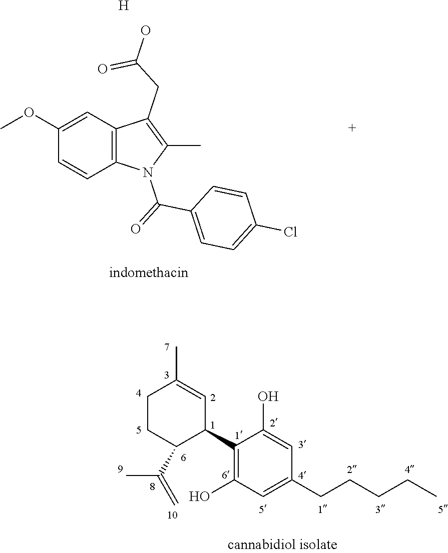

It is envisioned that the total dry volume of the combined formulations for a single dosage tablet or capsule comprises 50 mg of indomethacin and 25 mg of CBD isolate. It is envisioned that a single dosage tablet or capsule may comprise lower or higher dry volume quantities of indomethacin. However, a single dosage tablet or capsule has a maximum dry volume of 75 mg of indomethacin. It is envisioned that adjustments from the 50 mg (indomethacin) baseline includes an upward or downward proportional adjustment of the dry volume of CBD isolate, including for dry volume in intermediate ranges between the 25 mg and 75 mg maximum for indomethacin.

It is to be understood that the embodiments and claims are not limited in its application to the details of construction and arrangement of the components set forth in the description and illustrated in the drawings. Rather, the description and the drawings provide examples of the embodiments envisioned, but the claims are limited to the specific embodiments. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the U.S. Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way. It is intended that the application is defined by the claims appended hereto.

What is claimed is:

1. A process for forming a pharmaceutical tablet comprising the step of selecting one or more active ingredients from the group comprising an anti-inflammatory active ingredient, an analgesic active ingredient, an NSAID active ingredient, and an antipyretic active ingredient; combining the one or more active ingredients with cannabidiol isolate active ingredient, the process further comprising the steps of blending, granulating, drying, compressing, coating, and polishing the tablet.

2. A pharmaceutical composition comprising:
cannabidiol isolate, present in an amount ranging from about 2% to about 35% by dry weight of the composition;
one or more active ingredients selected from the group comprising naproxen, aspirin, diclofenac, celecoxib, etoricelecoxib, mefenamic acid, and indomethacin present in an amount ranging from about 65% to about 98% by dry weight of the composition; and
one or more pharmaceutically acceptable excipients, stabilizers, or carriers, selected from the group consisting of starch, sucrose, gelatin, sugar alcohols, isomalt, cellulose derivatives, and polyvinylpyrrolidone, wherein the composition is formulated for oral administration.

3. The pharmaceutical composition of claim 2, wherein the dosage form is by tablet form.

4. The pharmaceutical composition of claim 2, wherein the dosage form is by capsule form.

5. A method of treating pain in a patient in need thereof, comprising administering to the patient an effective amount of cannabidiol isolate and one or more of one or more active ingredients selected from the group comprising naproxen, aspirin, diclofenac, celecoxib, etoricelecoxib, mefenamic acid, and indomethacin, wherein cannabidiol isolate comprises 2% to 35% of the total weight of the active ingredient combination and the one or more of one or more active ingredients selected from the group comprising naproxen, aspirin, diclofenac, celecoxib, etoricelecoxib, mefenamic acid, and indomethacin comprises 65% to 98% of the total weight of the active ingredient combination, via oral administration.
